(19) United States
(12) Patent Application Publication
(10) Pub. No.: US 2013/0123485 A1
(43) Pub. Date: May 16, 2013

(54) CATIONIC LIPIDS, METHODS FOR PREPARING THE SAME, AND DELIVERY SYSTEMS HAVING ABILITY TO TRANSITION INTO CELLS COMPRISING THE SAME

(71) Applicants: Myung-Ok Park, Nowon-gu (KR); Eun Young Yoon, Gwanak-gu (KR)

(72) Inventors: Myung-Ok Park, Nowon-gu (KR); Eun Young Yoon, Gwanak-gu (KR)

(21) Appl. No.: 13/810,650

(22) PCT Filed: Jul. 12, 2011
(86) PCT No.: PCT/KR2011/005088

§ 371 Date: Jan. 16, 2013

(30) Foreign Application Priority Data
Jul. 18, 2010 (KR) 10-2010-0069276

(51) Int. Cl. C07K 5/06; C07C 233/47

(52) U.S. Cl. CPC C07K 5/06; C07C 233/47
USPC 536/24.5; 536/23.1; 536/29.1; 548/338.1; 554/56; 554/57

(57) ABSTRACT

The present invention provides cationic lipids, methods for preparing the same, and delivery systems comprising the same. The present invention can provide cationic lipids which enhance the efficiency of intracellular or in vivo delivery of multiple-anionic target compounds such as drugs, anticancer agents, nucleic acids, etc., have no intracellular toxicity, but show increased stability, methods for preparing the same, and delivery systems comprising the same.

### TECHNICAL FIELD

The present invention relates to cationic lipids comprising basic amino acids and their derivatives, methods for preparing the same, and delivery systems having the ability to transition into cells comprising the same, and more particularly to cationic lipids which have no intracellular toxicity, but have high intracellular transport efficiency and increased stability, methods for preparing the same, and delivery systems comprising the same.

Particularly, the present invention relates to cationic lipids capable of various modifications for improving physical, chemical and physiological characteristics, methods for preparing the same, and intracellular or in vivo delivery systems comprising the same, and relates to cationic lipids which are used for intracellular or in vivo delivery of a target material comprising multiple-anionic compounds such as polynucleotides and the like, and which are allowed to include hydrophilic polymer chains and/or targeting ligands, thereby increasing its half-life in the body or having target cell specificity, methods for preparing the same, and delivery systems comprising the same.

### BACKGROUND ART

The cell membrane is a semi-permeable lipid bilayer and acts as a physical barrier between the intracellular components and the extracellular environment. The cell membrane exhibits selective permeability and controls whether certain substances can be allowed to either enter or leave the cell. While small molecules or fat-soluble substances, that is, hydrophobic and non-polar substances can pass rapidly through the lipid bilayer and diffuse within the cell, charged molecules, that is, ions are difficult to pass through the cell membrane. Especially, since peptides, proteins, oligonucleotides, DNAs, RNAs, etc. which are the objects of interest in development of new drugs have charges, they are difficult to deliver into the cell. Eventually, this makes them difficult to use for therapeutic purposes.

In this regard, in recent years, development of vectors or carriers has been actively carried out for delivery of proteins, peptides, sugars, etc. into the body. In addition, as medicinal uses of various nucleic acid substances such as DNAs, siRNAs, miRNAs (microRNAs), antisense oligonucleic acids, etc. have been identified, development of delivery systems which deliver those substances into cells has become a significant part.

Viral vector is a very excellent technology to introduce nucleic acids into cells and adenoviral vectors, retroviral vector, etc. have been widely used for the intracellular delivery of genetic materials for research purposes and clinical tests for gene therapy purposes are currently underway. However, the use of viral vectors for gene therapy involves potential safety problems.

In addition, lipofections using cationic lipids have been widely used to deliver oligonucleotides, plasmid DNAs, RNAs, proteins, etc. into cells. Artificially synthesized cationic lipids form complexes with negative charged biomolecules such as DNAs, proteins, etc. and make these molecules deliver into cells. However, lipofections are susceptible to the presence or absence of serum or antibiotics in cell culture medium and have disadvantages: they exhibit a decline in delivery efficiency and cytotoxicity.

As stated above, the use of cationic lipids, that is, derivatives of lipids with a positively charged ammonium or sulfonium ion-containing headgroup for the delivery of negatively charged biomolecules, such as oligonucleotides and DNA segments as liposomal lipids has been widely reported. The positively charged headgroups of lipids interact with negatively charged cell surfaces and make the delivery of biomolecules to cells easier. Cationic lipids form complexes with anionic nucleic acid substances through stable ionic bonds and these complexes are transported by cell membrane fusion or intracellular endocytosis into cells.

Meanwhile, previously developed cationic lipids are compounds having primary to quaternary amines. Examples of these cationic lipids include N-[1-(2,3-dioleyloxy)propyl]-N,N,N-trimethylammonium chloride (DOTMA) and also include 1,2-bis(oleyloxy)3-3-(trimethylammonio)propane (DOTAP), 1,2-bis(dimyristoyloxy)3-3-(trimethylammonio)propane (DMTAP), 1,2-dimyristyloxypropyl-2-dimethylhydroxyethylammonium bromide (DMRIE), etc.

However, there is a report that the above lipids have relatively high gene transfer efficiency, but they have cytotoxicity. To overcome this cytotoxicity, lipids using amino acid linkers instead of non amino acid linkers have been synthesized. Researchers such as Quay et al. described cationic, neutral, and anionic lipids which were synthesized using various amino acids in US2008/0317839 A1. In addition, Korean Patent No. 10-0807060 reported that synthesis of cationic lipids by binding an anionic amino acid to an amine group of a fatty acid amine derivative can enhance the intracellular delivery of nucleic acid drugs. Furthermore, Korean Patent No. 10-0909786 disclosed cationic lipids of which delivery efficiency of oligonucleic acid is improved by binding a fatty acid amine to an amino acid region to which three to six lysines are combined.

In addition, WO2005/032593 provided a liposome having intracellular or nuclear entry ability and it provided cationic lipids to which polyamino acids having cationic groups including arginine residues are combined. However, these also still have concerns for cytotoxicity due to excessive cationic amino acid complexes.

Meanwhile, according to a recent report, many cationic lipids manufactured by binding fatty acid amines with carboxyl groups of amino acids had cytotoxicity contrary to expectations, and particularly, it was reported that most of the manufactured cationic lipids showed very low intracellular delivery efficiency of target materials such as oligonucleic acids, etc. and had no practical value. This suggests that since it is difficult to achieve the intracellular delivery efficiency with the formation of lipid delivery systems only by simple binding of amino acids and fatty acid amines and the delivery efficiency is determined according to their specific structures, they can be used practically as delivery systems only if a very meticulous prior design and experiments result support them (Akin Akinc et al., A combinatorial library of lipid-like materials for delivery of RNAi therapeutics, Nature Biotechnology, 2008, vol. 26, No. 5, pp 561-569).

Alternatively, the structure of liposomes which are microsomes comprising a lipid bilayer is similar to that of cell membranes, and liposomes have an advantage to deliver drugs easily through fusion with cells or endocytosis. However, the half-life of liposomes in the bloodstream is reduced rapidly over the administration time into the body due to easy absorption by reticuloendothelial system of spleen and macrophages, and liposomes become structurally unstable due to adsorption of blood proteins and coagulation of liposomes and thus this is becoming a problem to drug stability. To overcome this drawback, methods for increasing the half-life of liposomes in the body by introducing polyethylene glycol (hereinafter, referred to as “PEG”) to the surfaces of a phospholipid which is a component of liposomes and thus reducing the adsorption of blood proteins to liposomes have been proposed. However, the existing PEG-liposome complexes have a problem of lowering the intracellular transport.

Therefore, as stated above, liposomes of cationic lipids should be prepared considering the delivery efficiency of target materials and various intracellular metabolisms in the field of the invention, and it is necessary to develop liposomes of cationic lipids in a variety of ways and thus to achieve the development of modified liposomes which can improve physical, chemical, physiological characteristics of liposomes of cationic lipids.

That is, the existing methods for preparing cationic lipids and their structures have structural limits on modification of compounds for improving physical, chemical, and physiological characteristics, and therefore, it is necessary to develop new methods for preparing cationic lipids which allow various modifications to increase the intracellular delivery efficiency and half-life in cells, and cationic lipids having new structures obtained therefrom.

Accordingly, as a result of repeated studies, the present inventors have developed new methods for preparing new cationic lipids having high intracellular transport efficiency and increased stability and thus prepared new cationic lipid delivery systems. We have also synthesized cationic lipid delivery systems comprising targeting ligands, and thus, allowed them to be applied for the delivery of drugs requiring targeting.

### SUMMARY OF INVENTION

The object of the present invention is to provide cationic lipids which have no intracellular toxicity, but have high intracellular transport efficiency and increased stability, methods for preparing the same, and delivery systems having the ability to transition into cells comprising the same.

Specifically, the object of the present invention is to provide novel cationic lipids as described above, methods for preparing the same, and delivery systems comprising the same and to increase the intracellular or in vivo delivery efficiency of multiple-anionic target compounds such as anticancer agents, protein drugs, or nucleic acids.

The another object of the present invention is to provide cationic lipids which allow various modifications to improve their physical, chemical, and physiological characteristics, methods for preparing the same, and delivery systems comprising the same.

That is, the object of the present invention is to provide cationic lipids which can be used for the intracellular or in vivo delivery of target materials comprising multiple-anionic compounds such as anticancer agents, protein drugs, polynucleotides, etc. and comprise hydrophilic polymer chains and/or targeting ligands, methods for preparing the same, and delivery systems comprising the same. Specifically, the object of the present invention is to provide cationic lipid derivatives having increased half-life in the body or target cell specificity by binding cationic lipids with a biocompatible polymer of PEG, sugars such as galactose, mannose, glucose, and the like, or antibodies, as hydrophilic polymer chains or target-specific ligands.

### DETAILED DESCRIPTION OF INVENTION

Above all, the present invention provides a cationic lipid represented by the following Formula (I):

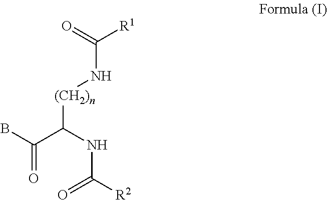

wherein n is 1 to 4, each of R¹ and R² is independently C7-C24 alkyl or alkenyl chain, and B is OH or A-NH, wherein A is a sugar or represented by the following Formula (II),

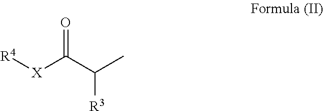

wherein X is NH or O, R³ is a hydrocarbon group having a cationic group derived from an amino acid and represented by the following Formulas (a), (b) and (c),

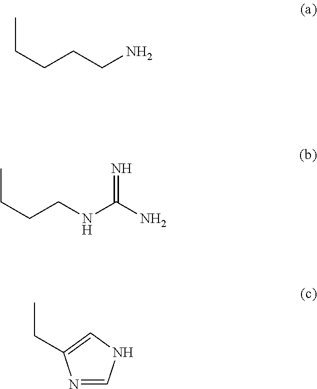

and R⁴ is a ligand and is alkyl, benzyl, a sugar, an antibody, polyethylene glycol, polypropylene glycol, or polyoxyethylene.

The present invention also provides a delivery system having the ability to transition into cells, comprising a cationic lipid represented by the following Formula (I):

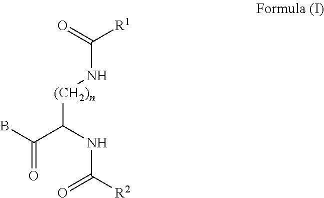

wherein n is 1 to 4, each of R¹ and R² is independently C7-C24 alkyl or alkenyl chain, and B is OH or A-NH, wherein A is a sugar or represented by the following Formula (II),

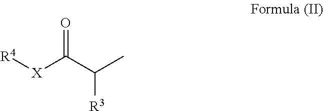

wherein X is NH or O, R³ is a hydrocarbon group having a cationic group derived from an amino acid and represented by the following Formulas (a), (b), and (c),

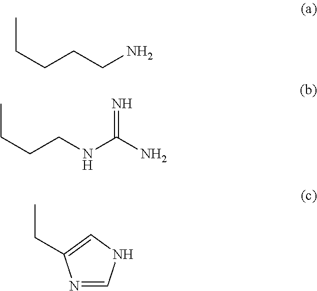

and R⁴ is a ligand and is alkyl or alkenyl, benzyl, a sugar, an antibody, polyethylene glycol, polypropylene glycol, or polyoxyethylene.

In the cationic lipid or delivery system comprising the same according to one embodiment of the present invention, each of R¹ and R² may be independently saturated or unsaturated hydrocarbon chain derived from stearate, laurate, myristate, palmitate, or oleate.

In the cationic lipid or delivery system comprising the same according to one embodiment of the present invention, R⁴ may be methyl, ethyl, propyl, isopropyl, n-butyl, or benzyl.

In the cationic lipid or delivery system comprising the same according to one embodiment of the present invention, the cationic lipid may use a biocompatible polymer such as mPEG (methoxy end-capped polyethylene glycol), polypropylene glycol, or polyoxyethylene as the ligand to increase the half-life in the body.

In the cationic lipid or delivery system comprising the same according to one embodiment of the present invention, the cationic lipid is formed by binding an amine group of an amino acid having a positive charge with a hydrophobic saturated or unsaturated fatty acid derivative, wherein a carbonyl group of a fatty acid halide, for example, a fatty acid chloride, is combined to the amine group of the amino acid. That is, in the conventional art, a fatty acid amine is combined to a carboxyl group of an amino acid, but, the mode of combination of an amino acid and a fatty acid derived hydrocarbon chain of the present invention is totally different from that of the conventional art.

In the case of the cationic lipid or delivery system comprising the same according to one embodiment of the present invention, a carboxyl group of an amino acid does not take part in the combination, so additional amino acid can be combined and various ligands can be combined thereto, and thus, there is an advantage that physical, chemical, physiological characteristics of the cationic lipid can be improved diversely.

Also, in the cationic lipid or delivery system comprising the same according to one embodiment of the present invention, the cationic lipid may use at least one sugar selected from the group consisting of mannitol, sorbitol, xylitol, glucitol, dulcitol, inositol, arabinitol, arabitol, galactitol, iditol, alitol, fructose, sorbose, glucose, mannose, xylose, trehalose, allose, dextrose, altrose, gulose, idose, galactose, talose, ribose, arabinose, lyxose, sucrose, maltose, lactose, lactulose, fucose, rhamnose, melezitose, maltotriose, and raffinose as the target cell specific ligand.

Meanwhile, the delivery system comprising the cationic lipid according to one embodiment of the present invention may comprise a drug or nucleic acid as a target material of intracellular or in vivo delivery. The drug may be an anticancer agent.

In the delivery system comprising the cationic lipid according to one embodiment of the present invention, the nucleic acid may be at least one nucleic acid selected from the group consisting of DNAs, RNAs, aptamers, siRNAs, miRNAs, and antisense oligonucleic acids.

In addition, in the delivery system comprising the cationic lipid according to one embodiment of the present invention, the drug may be at least one drug selected from the group consisting of ceftriaxone, ketoconazole, ceftazidime, oxaprozin, albuterol, valacyclovir, urofollitropin, famciclovir, flutamide, enalapril, mefformin, itraconazole, buspirone, gabapentin, fosinopril, tramadol, acarbose, lorazepan, follitropin, glipizide, omeprazole, fluoxetine, lisinopril, tramsdol, levofloxacin, zafirlukast, interferon, growth hormone, interleukin, erythropoietin, granulocyte stimulating factor, nizatidine, bupropion, perindopril, erbumine, adenosine, alendronate, alprostadil, benazepril, betaxolol, bleomycin sulfate, dexfenfluramine, diltiazem, fentanyl, flecainid, gemcitabine, glatiramer acetate, granisetron, lamivudine, mangafodipir trisodium, mesalamine, metoprolol fumarate, metronidazole, miglitol, moexipril, monteleukast, octreotide acetate, olopatadine, paricalcitol, somatropin, sumatriptan succinate, tacrine, verapamil, nabumetone, trovafloxacin, dolasetron, zidovudine, finasteride, tobramycin, isradipine, tolcapone, enoxaparin, fluconazole, lansoprazole, terbinafine, pamidronate, didanosine, diclofenac, cisapride, venlafaxine, troglitazone, fluvastatin, losartan, imiglucerase, donepezil, olanzapine, valsartan, fexofenadine, clacitonin, ipratropium bromide, adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, nicardipine hydrochloride, zolpidem tartrate, amlodipine besylate, ethinyl estradiol, omeprazole, rubitecan, amlodipine besylate/benazepril hydrochloride, etodolac, paroxetine hydrochlroride, atovaquone, podofilox, betamethasone dipropionate, pramipexole dihydrochloride, vitamin, quetiapine fumarate, candesartan, cilexetil, ritonavir, busulfan, carbamazepine, flumazenil, risperidone, carbemazepine, carbidopa, levodopa, ganciclovir, saquinavir, amprenavir, carboplatin, glyburide, sertraline hydrochloride, rofecoxib carvedilol, halobetasol propionate, sildenafil citrate, celecoxib, chlorthalidone, imiquimod, simvastatin, citalopram, ciprofloxacin, irinotecan hydrochloride, sparfloxacin, efavirenz, cisapride monohydrate, tamsulosin hydrochloride, mofafinil, azithromycin, clarithromycin, letrozole, terbinafine hydrochloride, rosiglitazone maleate, diclofenac sodium, lomefloxacin hydrochloride, tirofiban hydrochloride, telmisartan, diazapam, loratadine, toremifene citrate, thalidomide, dinoprostone, mefloquine hydrochloride, trandolapril, docetaxel, mitoxantrone hydrochloride, tretinoin, triamcinolone acetate, estradiol, nelfinavir mesylate, indinavir, beclomethasone dipropionate, famotidine, nifedipine, prednisone, cefuroxime, lorazepam, digoxin, lovastatin, griseofulvin, naproxen, ibuprofen, isotretinoin, tamoxifen citrate, nimodipine, amiodarone, and alprazolam.

In addition, in the delivery system comprising the cationic lipid according to one embodiment of the present invention, in the case that the drug is an anticancer agent, the anticancer agent may be at least one anticancer agent selected from the group consisting of paclitaxel, vinblastine, adriamycin, oxaliplatin, cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin, phenesterine, vincristine, tamoxifen, dasatinib, piposulfan, maytansinoid, taxanes and CC-1065.

Meanwhile, the present invention provides a method for preparing a cationic lipid of the following Formula (I), comprising (a) protecting an amine group (—NH₂) of an amino acid having a positive charge with a protecting group; (b) deprotecting the protected amine group to activate the amine group of the amino acid; and (c) binding a carbonyl group of a fatty acid halide to the activated amine group:

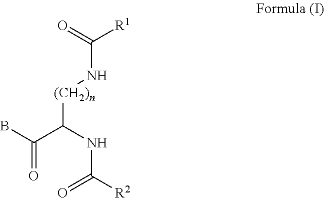

wherein n is 1 to 4, each of R¹ and R² is independently C7-C24 alkyl or alkenyl chain, and B is OH or A-NH, wherein A is a sugar or represented by the following Formula (II),

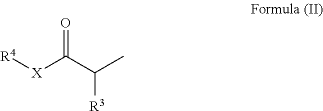

wherein X is NH or O, R³ is a hydrocarbon group having a cationic group derived from an amino acid and represented by the following Formulas (a), (b) and (c),

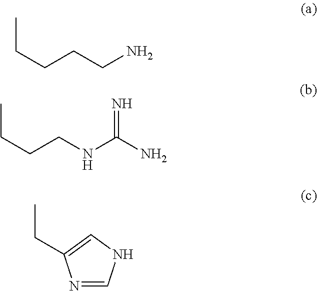

and R⁴ is a ligand and is alkyl, benzyl, a sugar, an antibody, polyethylene glycol, polypropylene glycol, or polyoxyethylene.

In the method for preparing the cationic lipid according to one embodiment of the present invention, in the step (a), the amine group (—NH₂) is protected with Boc protecting group using a solution in which tetrahydrofuran is added to t-(Boc)₂O, in the step (b), the protected amine group is deprotected using trifluoroacetate to activate the amine group of the amino acid, and in the step (c), the carbonyl group of the fatty acid halide is combined to the activated amine group using triethylamine. Preferably, the fatty acid halide may be a fatty acid chloride.

In the method for preparing the cationic lipid according to one embodiment of the present invention, each of R¹ and R² may be independently saturated or unsaturated hydrocarbon chain derived from stearate, laurate, myristate, palmitate, or oleate.

In addition, in the method for preparing the cationic lipid according to one embodiment of the present invention, to the carboxyl group of the amino acid portion of the cationic lipid, an amine group of another amino acid may be additionally combined to form an amide bond, or methyl, ethyl, propyl, isopropyl, n-butyl, benzyl, polyethylene glycol, polypropylene glycol, polyoxyethylene, or a sugar may be combined as a ligand, or an amine group of still another amino acid in which methyl, ethyl, propyl, isopropyl, n-butyl, benzyl, polyethylene glycol, polypropylene glycol, polyoxyethylene, or a sugar is combined to the carboxyl group of the still another amino acid as a ligand may be combined to form an amide bond.

In addition, in the method for preparing the cationic lipid according to one embodiment of the present invention, the sugar may be the ligand or target cell specific ligand selected from the group consisting of mannitol, sorbitol, xylitol, glucitol, dulcitol, inositol, arabinitol, arabitol, galactitol, iditol, alitol, fructose, sorbose, glucose, mannose, xylose, trehalose, allose, dextrose, altrose, gulose, idose, galactose, talose, ribose, arabinose, lyxose, sucrose, maltose, lactose, lactulose, fucose, rhamnose, melezitose, maltotriose, and raffinose.

Meanwhile, the delivery system comprising the cationic lipid according to one embodiment of the present invention may form a complex with a nucleic acid drug having an anionic charge such as plasmid genes or small interference RNAs due to charging properties, and can not only enhance the transport efficiency of target nucleic acid drugs into cells, but can also decrease the cytotoxicity, and thus, the delivery system can be used helpfully in the case that nucleic acid drugs are administered in vivo or intracellularly.

That is, the delivery system comprising the cationic lipid according to one embodiment of the present invention can provide a complex of a target material of delivery with the lipid delivery system, and since the delivery system comprising the cationic lipid according to one embodiment of the present invention having a formulation of liposomes, micelles, emulsions, or nanoparticles show cationic property, it can form an electrostatic complex with a negatively charged target material of delivery. Therefore, with the use of the delivery system comprising the cationic lipid according to one embodiment, a formulation process with anionic target materials of delivery can be convenient. Meanwhile, it may be easily understood by those skilled person in the art that formulations such as liposomes, micelles, emulsions, nanoparticles, etc. can be prepared using well known technology in the art.

Meanwhile, in the complex of the target material of delivery with the delivery system comprising the cationic lipid according to one embodiment of the present invention, the term “administration” means the introduction of the specified substances to a patient in any suitable way, and the administration route of the delivery system may be any general route as long as a drug can be arrived at its target tissue. Examples of the administration route may include, but are not limited to, intraperitoneal, intravenous, intramuscular, subcutaneous, intracutaneous, oral, topical, endonasal, intrapulmonary, intrarectal administration, etc. In addition, the complex of the target material of delivery with the delivery system comprising the cationic lipid according to one embodiment of the present invention may be administered by any equipment by which the active substance can move to a target cell. In addition, the therapeutically effective amount of the complex of the target material of delivery with the delivery system comprising the cationic lipid according to one embodiment of the present invention means an amount that is required for the administration to expect therapeutic effects on a disease of interest. Therefore, the therapeutically effect amount may be controlled depending on kinds of diseases, severity of diseases, kinds of target materials of delivery (drugs, antibiotics, or nucleic acids) to be administered, kinds of dosage forms age, gender, weight, general health status, diet, administration times, and administration methods. For example, when the complex of a drug with the delivery system comprising the cationic lipid is administered to an adult, a dose of 0.001 mg/kg to 100 mg/kg once a day may be administered.

### ADVANTAGEOUS EFFECTS

According to the present invention, cationic lipids which enhance the efficiency of intracellular or in vivo delivery of multiple-anionic target compounds such as drugs, anticancer agents, nucleic acids, etc., have no intracellular toxicity, but show increased stability, methods for preparing the same, and delivery systems comprising the same can be provided.

In addition, according to the present invention, by binding cationic lipids with a biocompatible polymer of polyethylene glycol (PEG), sugars such as galactose, mannose, glucose and the like, or antibodies, as hydrophilic polymer chains or target-specific ligands, the present invention can increase the half-life in the body or improve target cell specificity.

Therefore, the present invention will reinforce the intracellular transport efficiency of drugs such as DNAs, RNAs, aptamers, siRNAs, antisense oligonucleic acids, anticancer agents, etc., and increase the stability in the body and the ability for targeting drugs into specific cells due to the inclusion of target-specific ligands.

### BRIEF DESCRIPTION OF DRAWINGS

The above and other objects and features of the present invention will be more clearly understood to those skilled in the art from the following detailed description taken in conjunction with the accompanying drawings.

FIG. 1 is a photograph of images taken through a fluorescence microscope using fluorescent-labeled double stranded ribonucleic acid in the mouse hepatoma cell line Hepa 1-6 showing intracellular delivery of double stranded ribonucleic acid when treated with the complex form with the cationic liposome prepared in Comparative example 1 (B); when treated with the complex form with the cationic liposome containing mPEG-DSPE prepared in Comparative example 2 (C); and when treated with the complex forms with the liposome formulations containing the cationic lipids of the present invention prepared in Examples 23 (D), 24 (E), 25 (F), and 26 (G). For reference, FIG. 1 (A) is a fluorescence microscope of a control, conducted using the conventional commercially available LipofectAMINE 2000.

FIG. 2 is a photograph of images taken through a fluorescence microscope using fluorescent-labeled double stranded ribonucleic acid in the human lung carcinoma cell line A549 showing delivery of double stranded ribonucleic acid when treated with the complex form with the conventional cationic liposome prepared in Comparative example 1 (A) and when treated with the complex form with the liposome formulation containing the cationic lipid of the present invention prepared in Example 23 (B).

FIG. 3 is a photograph of images taken through a fluorescence microscope using fluorescent-labeled double stranded ribonucleic acid in the human kidney cell line 293T showing delivery of double stranded ribonucleic acid when treated with the complex form with the conventional cationic liposome prepared in Comparative example 1 (A) and when treated with the complex form with the liposome formulation containing the cationic lipid of the present invention prepared in Example 23 (B).

FIG. 4 is a graph showing the toxicity of complexes of siRNA with the cationic lipid-containing liposomes prepared in Examples 23 and 24 in Hepa 1-6, A549, and 293T cells.

FIG. 5 is a photograph of electrophoresis showing stability experiment results for individual complexes of ribonucleic acid with liposomes (A, B, C) comprising the cationic lipids of the present invention prepared in Examples 23, 24, 25 and complexes of ribonucleic acid with liposomes (D, E) prepared in Comparative examples 1, 2 in serum.

### EXAMPLES

The present invention provides the method for preparing novel cationic lipid delivery systems, and also provides the method for preparing cationic lipid delivery systems having target specific ligands. Prepared cationic lipid delivery systems provide liposome preparations which transport nucleic acids, anticancer agents, drugs, etc. efficiently into cells.

Hereinafter, the present invention is explained in detail in accordance with examples which do not limit the present invention. The following examples of the present invention are provided only for illustrating the present invention and are not intended to restrict or limit the scope of the present invention. Therefore, it is to be understood that what can be inferred easily from the detailed descriptions and examples of the present invention by those skilled in the art should belong to the scope of the present invention. References cited in the present specification are incorporated into the present invention.

### Preparation Example

### Cationic Lipid Synthesis Process

### Example 1

### Nα,Nε-distearoyl-lysine; 2,6-bis(stearamido)hexanoic acid Synthesis

### Example 1-1

14 mL of tetrahydrofuran was added to t-(Boc)₂O (3.57 g, 16.36 mmol) and stirred. Lysine monohydrochloride (1.3 g, 7.12 mmol) was added thereto, and 14 mL of 1 N sodium hydroxide was added thereto, followed by reaction at room temperature overnight. After the reaction was completed, tetrahydrofuran was concentrated under reduced pressure, and the residue was extracted with dichloromethane and the dichloromethane layer was removed, and the water layer was acid-treated with 1 N HCl solution to adjust the pH to 3-4 and extracted with dichloromethane. The extract was dried over anhydrous magnesium sulfate, filtered and concentrated.

### Example 1-2

The reaction product obtained in Example 1-1 was dissolved in 30 mL of dichloromethane, and 10 mL of trifluoroacetic acid was added dropwise thereto in an ice bath. The ice bath was removed, followed by reaction at room temperature for 6 hr, and after the reaction was completed, dichloromethane was concentrated under reduced pressure, and trifluoroacetic acid was removed by drying under vacuum.

### Example 1-3

The reaction product obtained in Example 1-2 was dissolved in 70 mL of acetone, and triethylamine (9.9 mL, 71.08 mmol) was added slowly thereto in an ice bath, followed by reaction for 30 min Stearoyl chloride (7.17 mL, 21.32 mmol) was slowly added dropwise thereto and the temperature was increased slowly to room temperature, followed by reaction overnight. Salt was removed by filtering and the filtrate was concentrated under reduced pressure and dichloromethane and water were added thereto and the mixture was acid-treated with 1 N HCl solution to adjust the pH to 3-4 and extracted with dichloromethane. The extract was dried over anhydrous magnesium sulfate, filtered and concentrated, and then, the residue was separated by column chromatography (dichloromethane:methanol=10:1) and recrystallized with hexane.

¹H NMR: (CDCl₃,300 MHz) δ 4.48 (m, 1H), 3.22 (m, 2H), 2.22 (m, 4H), 1.79 (m, 2H), 1.60 (m, 6H), 1.25 (m, 58H), 0.88 (t, 6H)

A reaction process of Example 1 is given in Reaction Formula 1 as below.

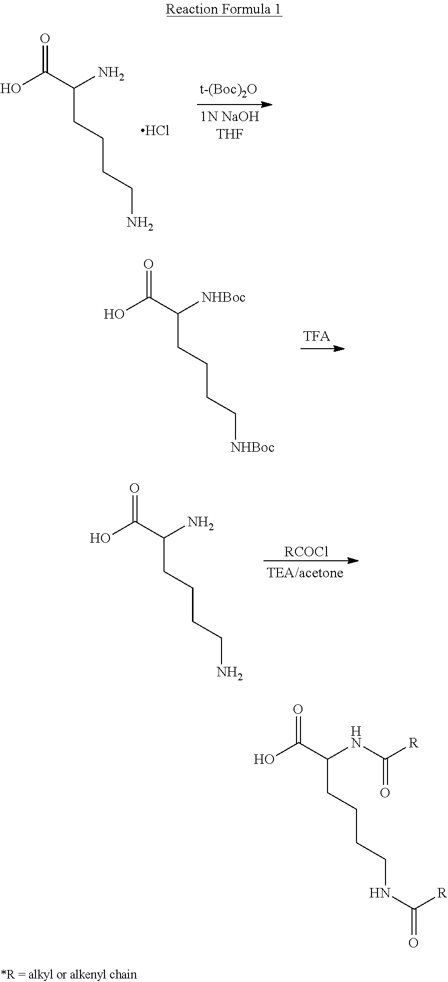

In Reaction Formula 1, each of R is independently C7-C24 alkyl or alkenyl chain and may be saturated or unsaturated hydrocarbon.

### Example 2

### Nα,Nε-dioleoyl-lysine; 2,6-bis(octadec-9-enamido)hexanoic acid Synthesis

The reaction product obtained in Example 1-2 was dissolved in 70 mL of acetone, and triethylamine (9.9 mL, 71.08 mmol) was added slowly thereto in an ice bath, followed by reaction for 30 min. Oleoyl chloride (8.3 mL, 21.33 mmol) was slowly added dropwise thereto and the temperature was increased slowly to room temperature, followed by reaction overnight. Salt was removed by filtering and the filtrate was concentrated under reduced pressure and dichloromethane and water were added thereto and the mixture was acid-treated with 1 N HCl solution to adjust the pH to 3-4 and extracted with dichloromethane. The extract was dried over anhydrous magnesium sulfate, filtered and concentrated, and then, the residue was separated by column chromatography (dichloromethane:methanol=10:1) and recrystallized with hexane.

¹H NMR: (CDCl₃,300 MHz) δ 5.32 (m, 4H), 4.50 (m, 1H), 3.27 (m, 2H), 2.20 (m, 4H), 2.01 (m, 8H), 1.8 (m, 2H), 1.60 (m, 6H), 1.28 (m, 42H), 0.86 (t, 6H)

### Example 3

### Nα,Nε-dioctanoyl-lysine; 2,6-bis(octanamido)hexanoic acid synthesis

The reaction product obtained in Example 1-2 was dissolved in 70 mL of acetone, and triethylamine (9.9 mL, 71.08 mmol) was added slowly thereto in an ice bath, followed by reaction for 30 min. Octanoyl chloride (3.7 mL, 21.46 mmol) was slowly added dropwise thereto and the temperature was increased slowly to room temperature, followed by reaction overnight. Salt was removed by filtering and the filtrate was concentrated under reduced pressure and dichloromethane and water were added thereto and the mixture was acid-treated with 1 N HCl solution to adjust the pH to 3-4 and extracted with dichloromethane. The extract was dried over anhydrous magnesium sulfate, filtered and concentrated, and then, the residue was separated by column chromatography (dichloromethane:methanol=10:1) and recrystallized with hexane.

### Example 4

### Nα,Nε-dilauroyl-lysine; 2,6-bis(dodecanamido)hexanoic acid Synthesis

The reaction product obtained in Example 1-2 was dissolved in 70 mL of acetone, and triethylamine (9.9 mL, 71.08 mmol) was added slowly thereto in an ice bath, followed by reaction for 30 min. Lauroyl chloride (5.03 mL, 21.32 mmol) was slowly added dropwise thereto and the temperature was increased slowly to room temperature, followed by reaction overnight. Salt was removed by filtering and the filtrate was concentrated under reduced pressure and dichloromethane and water were added thereto and the mixture was acid-treated with 1 N HCl solution to adjust the pH to 3-4 and extracted with dichloromethane. The extract was dried over anhydrous magnesium sulfate, filtered and concentrated, and then, the residue was separated by column chromatography (dichloromethane:methanol=10:1) and recrystallized with hexane.

### Example 5

### Nα,Nε-dimyristoyl-lysine; 2,6-bis(tetradecanamido)hexanoic acid Synthesis

The reaction product obtained in Example 1-2 was dissolved in 70 mL of acetone, and triethylamine (9.9 mL, 71.08 mmol) was added slowly thereto in an ice bath, followed by reaction for 30 min. Myristoyl chloride (6.0 mL, 21.41 mmol) was slowly added dropwise thereto and the temperature was increased slowly to room temperature, followed by reaction overnight. Salt was removed by filtering and the filtrate was concentrated under reduced pressure and dichloromethane and water were added thereto and the mixture was acid-treated with 1 N HCl solution to adjust the pH to 3-4 and extracted with dichloromethane. The extract was dried over anhydrous magnesium sulfate, filtered and concentrated, and then, the residue was separated by column chromatography (dichloromethane:methanol=10:1) and recrystallized with hexane.

### Example 6

### Nα,Nε-dipalmitoyl-lysine; 2,6-bis(palmitamido)hexanoic acid Synthesis

The reaction product obtained in Example 1-2 was dissolved in 70 mL of acetone, and triethylamine (9.9 mL, 71.08 mmol) was added slowly thereto in an ice bath, followed by reaction for 30 min. Palmitoyl chloride (6.6 mL, 21.32 mmol) was slowly added dropwise thereto and the temperature was increased slowly to room temperature, followed by reaction overnight. Salt was removed by filtering and the filtrate was concentrated under reduced pressure and dichloromethane and water were added thereto and the mixture was acid-treated with 1 N HCl solution to adjust the pH to 3-4 and extracted with dichloromethane. The extract was dried over anhydrous magnesium sulfate, filtered and concentrated, and then, the residue was separated by column chromatography (dichloromethane:methanol=10:1) and recrystallized with hexane.

### Example 7

### Nα,Nε-dibehenoyl-lysine; 2,6-bis(docosanamido)hexanoic acid Synthesis

The reaction product obtained in Example 1-2 was dissolved in 70 mL of acetone, and triethylamine (9.9 mL, 71.08 mmol) was added slowly thereto in an ice bath, followed by reaction for 30 min. Behenoyl chloride (7.7 g, 21.45 mmol) was slowly added dropwise thereto and the temperature was increased slowly to room temperature, followed by reaction overnight. Salt was removed by filtering and the filtrate was concentrated under reduced pressure and dichloromethane and water were added thereto and the mixture was acid-treated with 1 N HCl solution to adjust the pH to 3-4 and extracted with dichloromethane. The extract was dried over anhydrous magnesium sulfate, filtered and concentrated, and then, the residue was separated by column chromatography (dichloromethane:methanol=10:1) and recrystallized with hexane.

### Example 8

### Nα,Nε-dioleoyl-Dap; 2,3-bis(octadec-9-enamido)propanoic acid Synthesis

### Example 8-1

2,3-diaminopropionic acid monohydrochloride (1 g, 7.11 mmol) was reacted in accordance with the same methods as Example 1-1 and Example 1-2 to obtain 2,3-diaminopropionic acid.

### Example 8-2

The reaction product obtained in Example 8-1 was reacted in accordance with the same method as Example 2 to obtain Nα,Nε-dioleoyl-Dap.

¹H NMR: (CDCl₃,300 MHz) δ 5.34 (m, 4H), 4.33 (m, 1H), 3.99 (m, 2H), 2.27 (m, 4H), 2.01 (m, 8H), 1.62 (m, 4H), 1.28 (m, 40H), 0.87 (t, 6H)

A reaction process of Example 8 is given in Reaction Formula 2 as below.

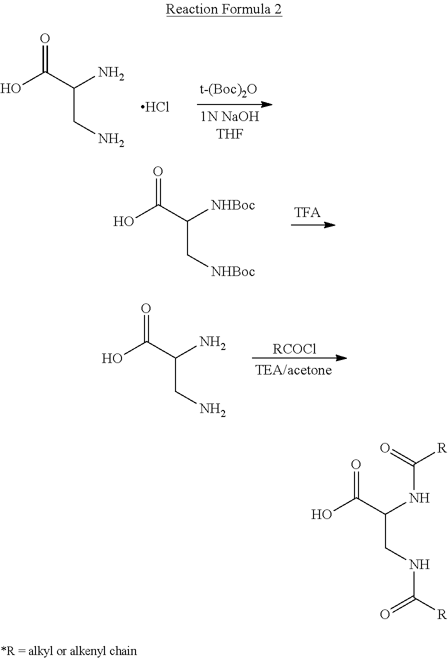

In Reaction Formula 2, each of R is independently C7-C24 alkyl or alkenyl chain and may be saturated or unsaturated hydrocarbon.

### Example 9

### mPEG-Arg-Lys-distearoyl Synthesis

### Example 9-1

15 mL of methanol was added to mPEG-NH₂ (1 g, 0.5 mmol) and PyBOP (390 mg, 0.75 mmol), HOBt (115 mg, 0.75 mmol) and stirred. Arginine (105 mg, 0.6 mmol) and 5 mL of water were added thereto respectively, followed by addition of diisopropylethylamine (0.26 mL, 1.49 mmol) and stirring overnight. After the reaction was completed, solvents were concentrated under reduced pressure and the residue was acid-treated with 1 N HCl solution to adjust the pH to 3-4 and extracted with dichloromethane. The extract was dried over anhydrous magnesium sulfate, filtered and concentrated, and then, the residue was separated and purified by column chromatography (dichloromethane:methanol=20:1).

### Example 9-2

4 mL of dichloromethane was added to the reaction product obtained in Example 1 (26 mg, 0.038 mmol) and PyBOP (33.5 mg, 0.064 mmol), HOBt (9.8 mg, 0.064 mmol) and stirred. Diisopropylethylamine (16.8 μL, 0.097 mmol) was added thereto in an ice bath, followed by reaction for 30 min, and then, the reaction product obtained in Example 9-1 (70 mg, 0.032 mmol) dissolved in 3 mL of dichloromethane was added thereto. After 10 min, the ice bath was removed and the mixture was stirred overnight. After the reaction was completed, solvents were concentrated under reduced pressure and the residue was acid-treated with 1 N HCl solution to adjust the pH to 3-4 and extracted with dichloromethane. The extract was dried over anhydrous magnesium sulfate, filtered and concentrated, and then, the residue was separated and purified by column chromatography (dichloromethane:methanol=10:1).

¹H NMR: (CDCl₃,300 MHz) δ 4.39 (m, 2H), 3.66 (m, 182H), 3.38 (s, 3H), 3.17 (m, 2H), 2.18 (m, 4H), 1.83 (m, 4H), 1.61 (m, 8H), 1.25 (m, 58H), 0.88 (t, 6H)

A reaction process of Example 9 is given in Reaction Formula 3 as below.

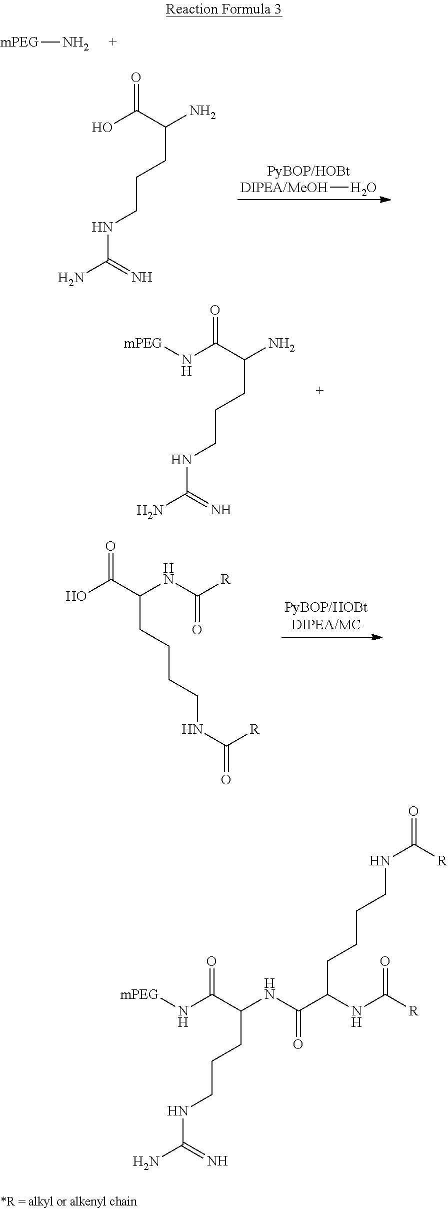

In Reaction Formula 3, each of R is independently C7-C24 alkyl or alkenyl chain and may be saturated or unsaturated hydrocarbon.

### Example 10

mPEG-Arg-Lys-dioleyl Synthesis

The reaction product obtained Example 2 (25 mg, 0.038 mmol) was reacted in accordance with the same method as Example 9-2 to obtain mPEG-Arg-Lys-dioleyl.

¹H NMR: (CDCl₃,300 MHz) δ 5.34 (m, 4H), 4.40 (m, 1H), 4.20 (m, 1H), 3.65 (m, 182H), 3.38 (s, 3H), 3.17 (m, 2H), 2.20 (m, 4H), 2.01 (m, 8H), 1.8 (m, 4H), 1.60 (m, 8H), 1.28 (m, 42H), 0.88 (t, 6H)

### Example 11

### mPEG-Arg-Dap-dioleyl Synthesis

The reaction product obtained Example 8 (24 mg, 0.038 mmol) was reacted in accordance with the same method as Example 9-2 to obtain mPEG-Arg-Dap-dioleyl.

¹H NMR: (CDCl₃,300 MHz) δ 5.33 (m, 4H), 4.40 (m, 1H), 3.71 (m, 182H), 3.38 (s, 3H), 2.21 (m, 4H), 2.01 (m, 10H), 1.62 (m, 6H), 1.28 (m, 40H), 0.87 (t, 6H)

A reaction process of Example 11 is given in Reaction Formula 4 as below.

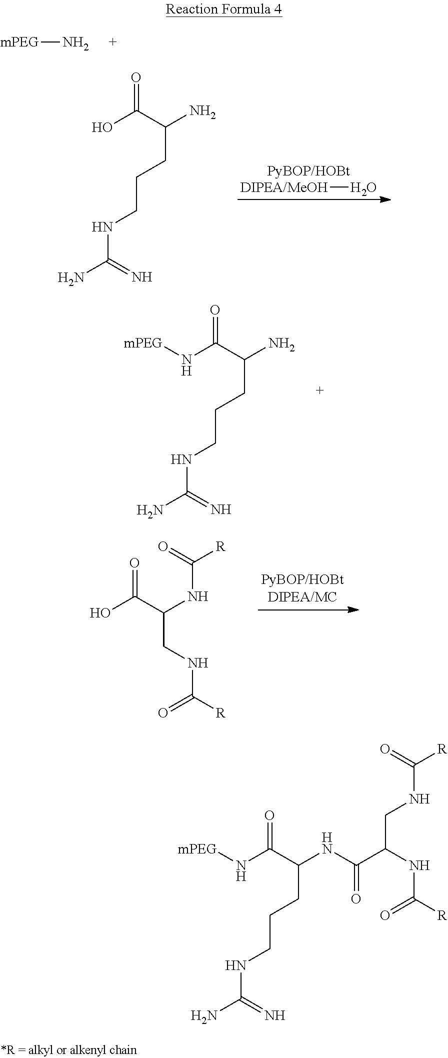

In Reaction Formula 4, each of R is independently C7-C24 alkyl or alkenyl chain and may be saturated or unsaturated hydrocarbon.

### Example 12

### MeO-Arg-Lys-dioleyl; 2-(2,6-Bis-octadec-9-enoylamino-hexanoyl amino)-5-guanidino-pentanoic acid methyl ester Synthesis

The reaction product obtained in Example 2 (26 mg, 0.038 mmol) and L-arginine methylester dihydrochloride (12 mg, 0.046 mmol) were reacted in accordance with the same method as Example 9-2 to obtain MeO-Arg-Lys-dioleyl.

¹H NMR: (CDCl₃,300 MHz) δ 5.33 (m, 4H), 4.55 (m, 1H), 4.40 (m, 1H), 3.68 (s, 3H), 3.28 (m, 4H), 2.23 (m, 4H), 2.01 (m, 8H), 1.80 (m, 2H), 1.70 (m, 2H), 1.60 (m, 8H), 1.28 (m, 42H), 0.86 (t, 6H)

A reaction process of Example 12 is given in Reaction Formula 5 as below.

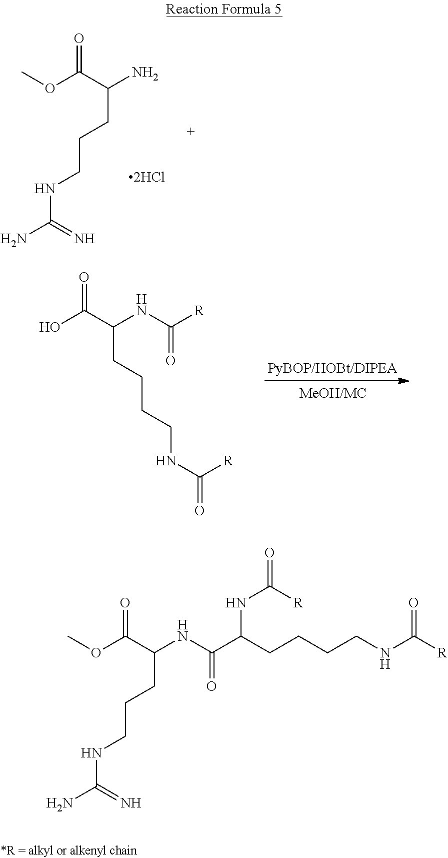

In Reaction Formula 5, each of R is independently C7-C24 alkyl or alkenyl chain and may be saturated or unsaturated hydrocarbon.

### Example 13

### MeO-Arg-Lys-dioctanyl Synthesis

The reaction product obtained in Example 3 (15 mg, 0.038 mmol) and L-arginine methylester dihydrochloride (12 mg, 0.046 mmol) were reacted in accordance with the same method as Example 9-2 to obtain MeO-Arg-Lys-dioctanyl.

### Example 14

### MeO-Arg-Lys-dilauryl Synthesis

The reaction product obtained in Example 4 (19.4 mg, 0.038 mmol) and L-arginine methylester dihydrochloride (12 mg, 0.046 mmol) were reacted in accordance with the same method as Example 9-2 to obtain MeO-Arg-Lys-dilauryl.

### Example 15

### MeO-Arg-Lys-dimyristyl Synthesis

The reaction product obtained in Example 5 (21.5 mg, 0.038 mmol) and L-arginine methylester dihydrochloride (12 mg, 0.046 mmol) were reacted in accordance with the same method as Example 9-2 to obtain MeO-Arg-Lys-dimyristyl.

### Example 16

### MeO-Arg-Lys-dipalmityl Synthesis

The reaction product obtained in Example 6 (24 mg, 0.038 mmol) and L-arginine methylester dihydrochloride (12 mg, 0.046 mmol) were reacted in accordance with the same method as Example 9-2 to obtain MeO-Arg-Lys-dipalmityl.

### Example 17

### MeO-Arg-Lys-dibehenyl Synthesis

The reaction product obtained in Example 7 (30 mg, 0.038 mmol) and L-arginine methylester dihydrochloride (12 mg, 0.046 mmol) were reacted in accordance with the same method as Example 9-2 to obtain MeO-Arg-Lys-dibehenyl.

### Example 18

### nBuO-Arg-Lys-dioleyl Synthesis

The reaction product obtained in Example 2 (26 mg, 0.038 mmol) and L-arginine n-butylester dihydrochloride (14 mg, 0.046 mmol) were reacted in accordance with the same method as Example 9-2 to obtain nBuO-Arg-Lys-dioleyl.

A reaction process of Example 18 is given in Reaction Formula 6 as below.

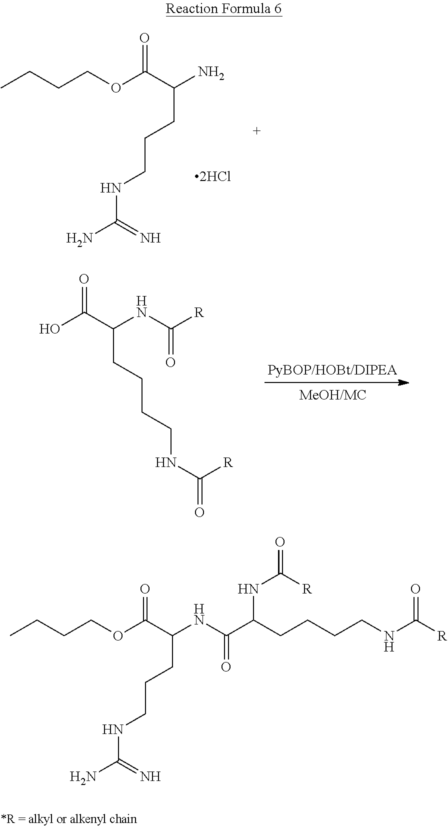

In Reaction Formula 6, each of R is independently C7-C24 alkyl or alkenyl chain and may be saturated or unsaturated hydrocarbon.

### Example 19

### MeO-His-Lys-dioleyl Synthesis

The reaction product obtained in Example 2 (26 mg, 0.038 mmol) and L-histidine methylester dihydrochloride (11.2 mg, 0.046 mmol) were reacted in accordance with the same method as Example 9-2 to obtain MeO-His-Lys-dioleyl.

A reaction process of Example 19 is given in Reaction Formula 7 as below.

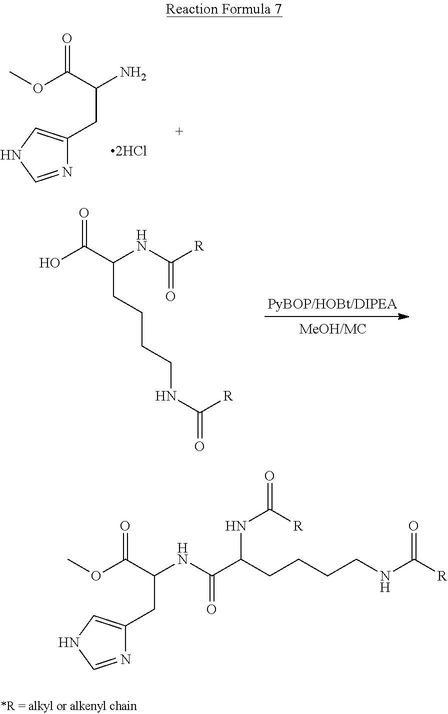

In Reaction Formula 7, each of R is independently C7-C24 alkyl or alkenyl chain and may be saturated or unsaturated hydrocarbon.

### Example 20

### MeO-Lys(Z)-Lys-dioleyl Synthesis

The reaction product obtained in Example 2 (26 mg, 0.038 mmol) and Nε-Z-L-lysine methylester hydrochloride (15.2 mg, 0.046 mmol) were reacted in accordance with the same method as Example 9-2 to obtain MeO-Lys(Z)-Lys-dioleyl.

A reaction process of Example 20 is given in Reaction Formula 8 as below.

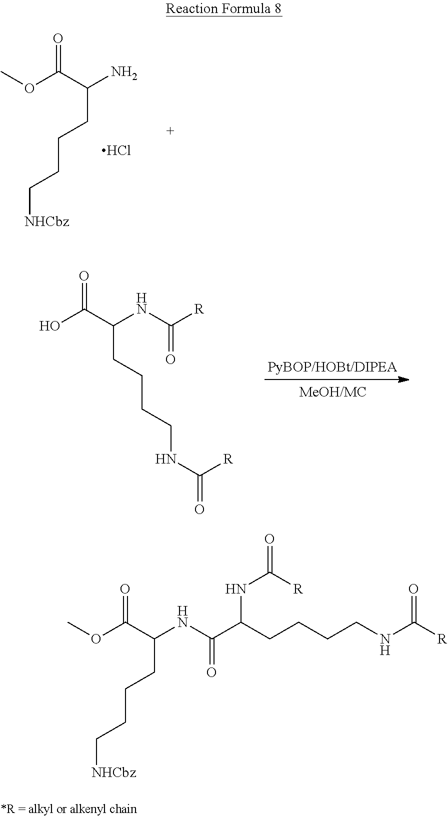

In Reaction Formula 8, each of R is independently C7-C24 alkyl or alkenyl chain and may be saturated or unsaturated hydrocarbon.

### Example 21

### Gal-Arg-Lys-dioleyl Synthesis

### Example 21-1

3 mL of DMF was added to Gal-NH₂ (53 mg, 0.2958 mmol) and PyBOP (307 mg, 0.5899 mmol), HOBt (90 mg, 0.5879 mmol) and stirred. Diisopropylethylamine (0.25 mL, 1.428 mmol) was added thereto, and then, Boc-Arg-OH (81 mg, 0.2953 mmol) was added thereto and the mixture was stirred overnight. After the reaction was completed, solvents were concentrated under reduced pressure and the residue was acid-treated with 1 N HCl solution to adjust the pH to 3-4 and extracted with dichloromethane. The extract was dried over anhydrous magnesium sulfate, filtered and concentrated, and then, the residue was separated and purified by column chromatography.

### Example 21-2

1 mL of 4M HCL in 1,4-dioxane was added to the reaction product obtained in Example 21-1 (60 mg, 0.1378 mmol) and stirred. After the reaction was completed, the solvent was concentrated under reduced pressure and ethylether was added to the residue. The residue was concentrated under reduced pressure to form a solid. The formed solid was dried under vacuum condition.

### Example 21-3

2 mL of DMF was added to the reaction product obtained in Example 2 (91 mg, 0.1348 mmol) and PyBOP (140 mg, 0.269 mmol), HOBt (41 mg, 0.2678 mmol) and stirred. Diisopropylethylamine (0.11 mL, 0.628 mmol) was added thereto, followed by reaction for 30 min, and then, the reaction product obtained in Example 21-2 (50 mg, 0.1345 mmol) was added thereto. After stirring overnight and the reaction was completed, the reaction product was acid-treated with 1 N HCl solution to adjust the pH to 3-4 and extracted with dichloromethane. The extract was dried over anhydrous magnesium sulfate, filtered and concentrated, and then, the residue was separated and purified by column chromatography.

A reaction process of Example 21 is given in Reaction Formula 9 as below.

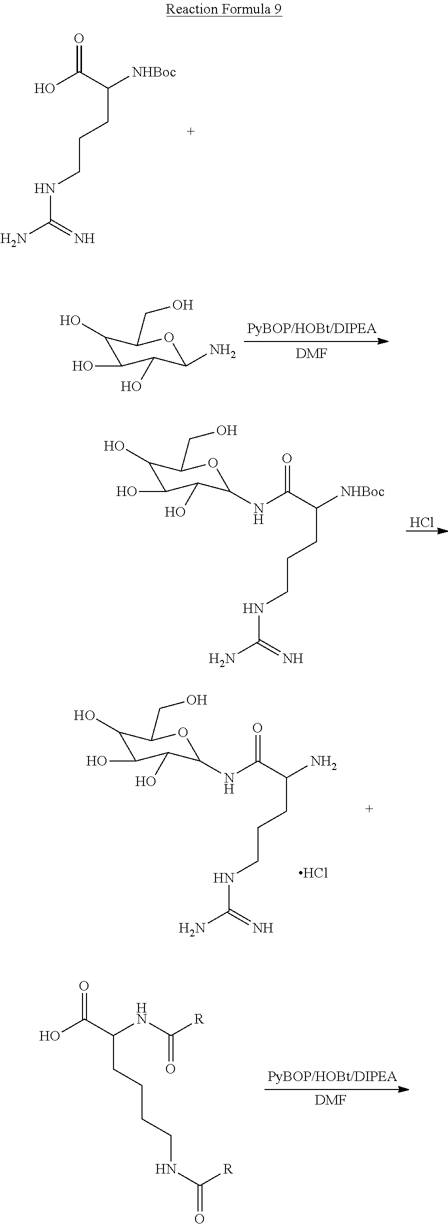

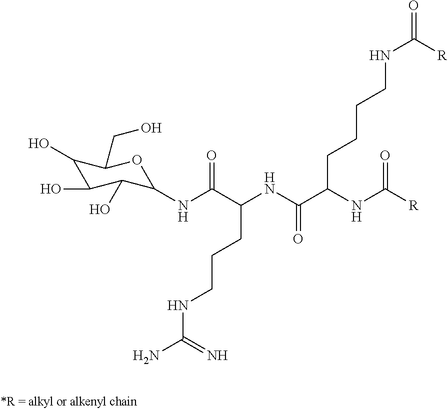

In Reaction Formula 9, each of R is independently C7-C24 alkyl or alkenyl chain and may be saturated or unsaturated hydrocarbon.

### Example 22

### Gal-Lys-dioleyl Synthesis

0.5 mL of DMF was added to the reaction product obtained in Example 2 (20 mg, 0.02963 mmol) and EDC.HCl (17 mg, 0.08868 mmol) and stirred. Gal-NH₂ (10 mg, 0.05581 mmol) was added thereto and stirred overnight. After the reaction was completed, the reaction product was acid-treated with 1 N HCl solution to adjust the pH to 3-4 and extracted with dichloromethane. The extract was dried over anhydrous magnesium sulfate, filtered and concentrated, and then, the residue was separated and purified by column chromatography (dichloromethane:methanol=20:1).

A reaction process of Example 22 is given in Reaction Formula 10 as below.

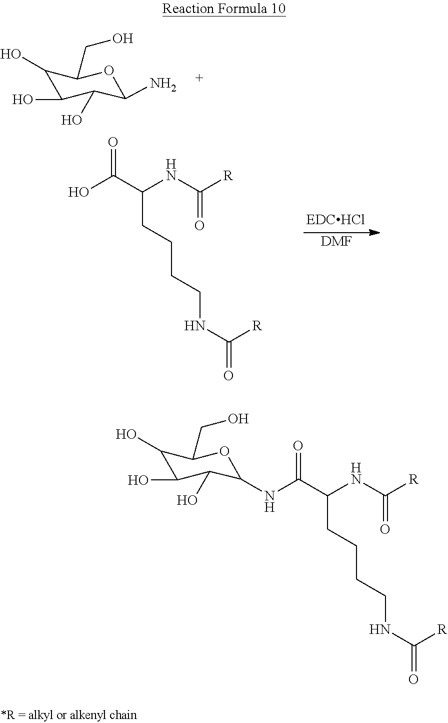

In Reaction Formula 10, each of R is independently C7-C24 alkyl or alkenyl chain and may be saturated or unsaturated hydrocarbon.

### Preparation Example of Delivery System Comprising Cationic Lipid

### Example 23

### Preparation of Cationic Liposome Containing MeO-Arg-Lys-dioleyl

The cationic lipid MeO-Arg-Lys-dioleyl prepared in Example 12, a cell-fusogenic phospholipid DOPE (Avanti Polar Lipid Inc., USA), and cholesterol (Avanti Polar Lipid Inc., USA) were dissolved in 1 mL of a solution of chloroform:methanol (3:1), respectively, and then, each of the resulting solutions was taken in a molar ratio of 1:1:1, added into a 10 mL glass septum vial and mixed, and then, rotary-evaporated at a low speed under nitrogen condition until the solution of chloroform:methanol was completely evaporated, thereby preparing a lipid thin film. For preparation of lipid multilamella vesicles, 1 mL of a phosphate-buffered solution was added to the thin film, and the vial was sealed at 37° C., followed by vortexing for 3 min. To obtain a uniform particle size, the vial solution was passed ten times through a 0.1 μm polycarbonate membrane using an extruder (Avanti Polar Lipid Inc., USA).

### Example 24

### Preparation of Cationic Liposome Containing MeO-Arg-Lys-dioleyl and mPEG-Arg-Dap-dioleyl

The cationic lipid MeO-Arg-Lys-dioleyl prepared in Example 12, the cationic lipid mPEG-Arg-Dap-dioleyl comprising a polyethylene glycol lipid derivative prepared in Example 11, a cell-fusogenic phospholipid DOPE (Avanti Polar Lipid Inc., USA), and cholesterol (Avanti Polar Lipid Inc., USA) were dissolved in 1 mL of a solution of chloroform:methanol (3:1), respectively, and then, each of the resulting solutions was taken in a molar ratio of 0.99:0.01:1:1, thereby preparing a cationic liposome in accordance with the same method as Example 23, and finally preparing a cationic liposome having a polyethylene glycol group present on a surface thereof.

### Example 25

### Preparation of Cationic Liposome Containing MeO-Arg-Lys-dioleyl and mPEG-DSPE

The cationic lipid MeO-Arg-Lys-dioleyl prepared in Example 12, mPEG-DSPE (Avanti Polar Lipid Inc., USA), a cell-fusogenic phospholipid DOPE (Avanti Polar Lipid Inc., USA), and cholesterol (Avanti Polar Lipid Inc., USA) were dissolved in 1 mL of a solution of chloroform:methanol (3:1), respectively, and then, each of the resulting solutions was taken in a molar ratio of 0.99:0.01:1:1, thereby preparing a cationic liposome in accordance with the same method as Example 23, and finally preparing a cationic liposome having a polyethylene glycol group present on a surface thereof.

### Example 26

### Preparation of Cationic Liposome Containing Gal-Lys-dioleyl

The cationic lipid MeO-Arg-Lys-dioleyl prepared in Example 12, the cationic lipid Gal-Lys-dioleyl prepared in Example 22, a cell-fusogenic phospholipid DOPE (Avanti Polar Lipid Inc., USA), and cholesterol (Avanti Polar Lipid Inc., USA) were dissolved in 1 mL of a solution of chloroform:methanol (3:1), respectively, and then, each of the resulting solutions was taken in a molar ratio of 0.95:0.05:1:1, thereby preparing a cationic liposome in accordance with the same method as Example 23.

### Comparative Example 1

### Preparation of Liposome Using Conventional Cationic Lipid

A cationic lipid DC-Chol (Avanti Polar Lipid Inc., USA), a cell-fusogenic phospholipid DOPE (Avanti Polar Lipid Inc., USA), and cholesterol (Avanti Polar Lipid Inc., USA) were dissolved in 1 mL of a solution of chloroform:methanol (3:1), respectively, and then, each of the resulting solutions was taken in a molar ratio of 1:1:1, added into a Pyrex 10 mL glass septum vial and mixed, and then, rotary-evaporated at a low speed under nitrogen condition until the solution of chloroform:methanol was completely evaporated, thereby preparing a lipid thin film. For preparation of lipid multilamella vesicles, 1 mL of a phosphate-buffered solution was added to the thin film, and the vial was sealed at 37° C., followed by vortexing for 3 min. To obtain a uniform particle size, the vial solution was passed ten times through a 0.1 μm polycarbonate membrane using an extruder (Avanti Polar Lipid Inc., USA).

### Comparative Example 2

### Preparation of Liposome Containing Conventional Cationic Lipid and mPEG-DSPE

A cationic lipid DC-Chol (Avanti Polar Lipid Inc., USA), mPEG-DSPE (Avanti Polar Lipid Inc., USA), a cell-fusogenic phospholipid DOPE (Avanti Polar Lipid Inc., USA), and cholesterol (Avanti Polar Lipid Inc., USA) were dissolved in 1 mL of a solution of chloroform:methanol (3:1), respectively, and then, each of the resulting solutions was taken in a molar ratio of 0.99:0.01:1:1, thereby preparing a cationic liposome in accordance with the same method as Comparative example 1 and finally preparing a cationic liposome having a polyethylene glycol group present on a surface thereof.

### Comparative Example 3

### Conventional Commercially Available Expression Reagent

LipofectAMINE 2000 (Invitrogen, USA), which is a conventional commercially available expression reagent, was purchased and used according to the manufacturer's instructions.

### Evaluation of Nucleic Acid Delivery Efficiency of Cationic Lipid-Containing Nucleic Acid Delivery Systems

### Example 27

### Evaluation of Nucleic Acid Delivery Efficiency Using Fluorescent Marker-Labeled siRNA

### Example 27-1

### Cell Culture

The mouse hepatoma cell line Hepa 1-6, the human lung carcinoma cell line A549, and the human kidney cell line 293T were purchased from American Type Culture Collection (ATCC, USA) to use. Hepa 1-6 and 293T cell lines were cultured in DMEM (Dulbecco's modified eagles medium, Gibco, USA) containing 10% w/v fetal bovine serum (Gibco, USA), 100 units/mL of penicillin and 100 μg/mL of streptomycin. A549 cell line was cultured in RPMI 1640 (Gibco, USA) comprising 10% fetal bovine serum, penicillin and streptomycin.

### Example 27-2

### Evaluation of Delivery Efficiency of siRNA in Hepa 1-6 Cell Line

On the day prior to the experiment, Hepa 1-6 cell line was seeded on 24-well plates at 8×10⁴ cells/well. When the cells of each plate were grown to 60% to 70% confluency, culture media of the plates were removed and fresh media were added to the plates at 500 μL/well. 50 μL of serum-free media were added to Eppendorf tubes. 2 μL of Block-iT (20 μmol, Invitrogen, USA), a fluorescent marker-labeled siRNA and 10 μL of each of cationic liposomes prepared in Comparative examples 1, 2, 3 and Examples 23, 24, 25, 26 were added to each of the Eppendorf tubes, respectively. These materials were slowly pipetted, mixed and allowed to incubate at room temperature for 20 min. The prepared complexes were added to the well plates, followed by cell culture in a CO₂ incubator at 37° C. for 24 hr. The cell-cultured media were replaced with fresh media at 500 μL/well, and then the gene transfer (transfection) efficiency was examined under a fluorescence microscope.

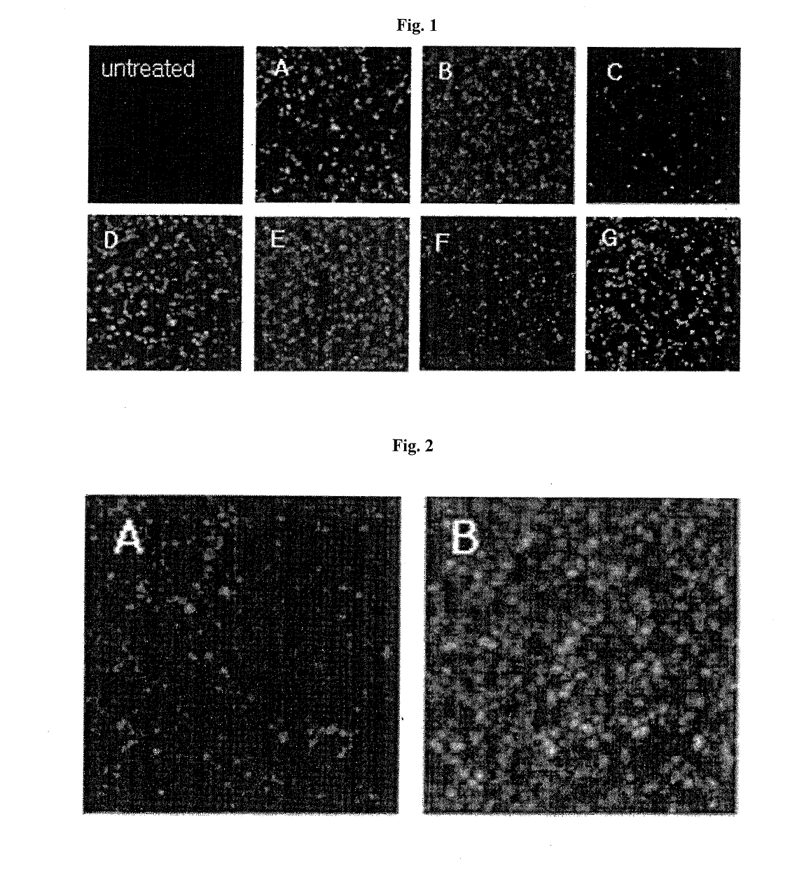

FIG. 1 is a photograph of images taken through a fluorescence microscope using fluorescent-labeled double stranded ribonucleic acid showing intracellular delivery of double stranded ribonucleic acid when treated with the complex form with the cationic liposome prepared in Comparative example 1 (B); when treated with the complex form with the cationic liposome containing mPEG-DSPE prepared in Comparative example 2 (C); and when treated with the complex forms with the liposome formulations containing the cationic lipids prepared in Examples 23 (D), 24 (E), 25 (F) and 26 (G).

From the results of FIG. 1, it can be seen that the cationic liposome containing the cationic lipid of the present invention prepared in Example 23 exhibits similar or increased delivery efficiency as compared with the expression reagent of Comparative example 3 (A) (used as a control) and exhibits much increased intracellular siRNA delivery efficiency as compared with the conventional liposome of Comparative example 1 (B).

In addition, it can be seen that the cationic liposome containing the PEG-conjugated cationic lipid of the present invention prepared in Example 24 (E) exhibits increased intracellular siRNA delivery efficiency as compared with the liposome containing the conventional lipid and PEG-DSPE prepared in Comparative example 2. Additionally, it can be seen that the cationic liposome containing the galactose-combined lipid of the present invention prepared in Example 26 exhibits increased intracellular siRNA delivery efficiency as compared with the cationic liposome prepared in Example 23.

### Example 27-3

### Evaluation of Delivery Efficiency of siRNA in A549 Cell Line

On the day prior to the experiment, A549 cell line was seeded on 24-well plates at 8×10⁴ cells/well. In accordance with the same method as Example 27-2, each complex of Block-iT with cationic liposomes prepared in Comparative example 1 and Example 23 was prepared, respectively and added to the well plates, followed by cell culture in a CO₂ incubator at 37° C. for 24 hr. The cell-cultured media were replaced with fresh media at 500 μL/well, and the nucleic acid transfer efficiency was examined under a fluorescence microscope.

FIG. 2 is a photograph of images taken through a fluorescence microscope using fluorescent-labeled double stranded ribonucleic acid in the human lung carcinoma cell line A549 showing delivery of double stranded ribonucleic acid when treated with the complex form with the conventional cationic liposome prepared in Comparative example 1 (A) and when treated with the complex form with the liposome formulation containing the cationic lipid of the present invention prepared in Example 23 (B). From the results of FIG. 2, it can be seen that the cationic liposome containing the cationic lipid of the present invention prepared in Example 23 exhibits increased siRNA delivery efficiency as compared with the conventional liposome prepared in Comparative example 1.

### Example 27-4

### Evaluation of Delivery Efficiency of siRNA in 293T Cell Line

On the day prior to the experiment, 293T cell line was seeded on 24-well plates at 8×10⁴ cells/well and in accordance with the same method as Example 27-2, each complex of Block-iT with cationic liposomes prepared in Comparative example 1 and Example 23 was prepared, respectively and the nucleic acid transfer efficiency was examined under a fluorescence microscope.

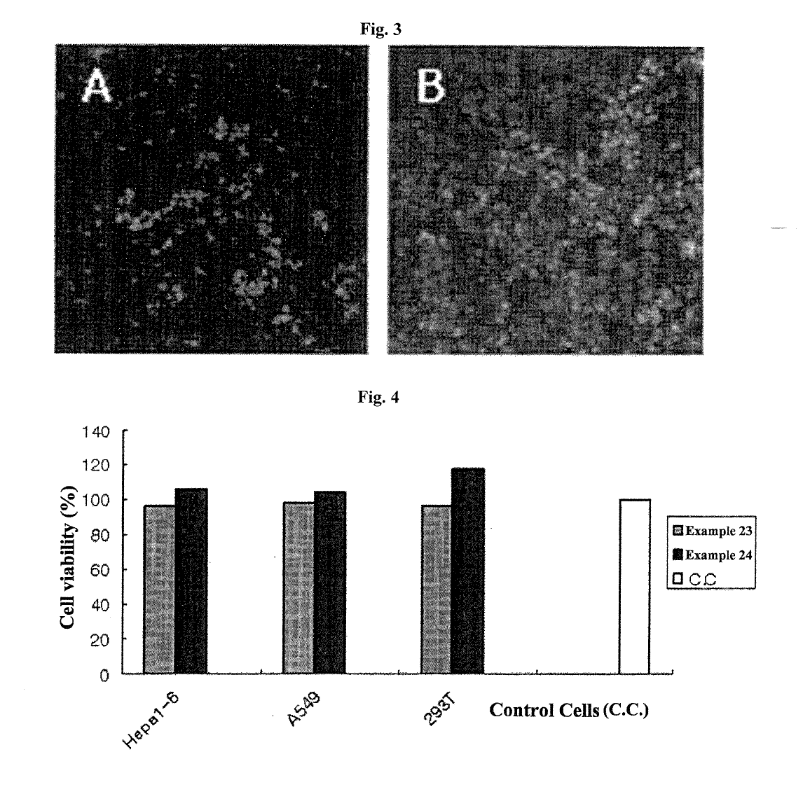

FIG. 3 is a photograph of images taken through a fluorescence microscope using fluorescent-labeled double stranded ribonucleic acid in the human kidney cell line 293T showing delivery of double stranded ribonucleic acid when treated with the complex form with the conventional cationic liposome prepared in Comparative example 1 (A) and when treated with the complex form with the liposome formulation containing the cationic lipid of the present invention prepared in Example 23 (B). From the results of FIG. 3, it can be seen that the cationic liposome containing the cationic lipid of the present invention prepared in Example 23 exhibits increased siRNA delivery efficiency as compared with the conventional liposome prepared in Comparative example 1.

### Example 28

### Toxicity Evaluation of Cationic Lipid-Containing Nucleic Acid Delivery Systems

### Example 28-1

### Toxicity Evaluation of Cationic Lipid-Containing Nucleic Acid Delivery Systems on Hepa 1-6 Cell Line

For evaluation of cytotoxicity of nucleic acid delivery systems comprising the novel cationic lipids of the present invention, the experiment was carried out according to the following procedures.

The mouse hepatoma cell line Hepa 1-6 was treated with cationic lipid-containing liposomes prepared in Examples 23 and 24 and the cytotoxicity was evaluated.

The cytotoxicity was evaluated by 3-(4,5-dimethylthiazol-2-yl)-2,5-diphenyl tetrazolium bromide (MTT) reagent assay.

The cells were seeded onto 96-well plates at 2×10⁴ cells/well, cultured for 12 hr and treated with cationic lipid-containing liposomes prepared in Examples 23 and 24, respectively. After incubating for 24 hr, MTT solution was added to make 10% of the culture media, followed by cell culture for another 4 hr. Then, the supernatant was removed and 0.04 N isopropanol hydrochloride solution was added to the media. Then, absorbance values were measured at 540 nm using an ELISA reader. Non-treated cells were used as a control.

### Example 28-2

### Toxicity Evaluation of Cationic Lipid-Containing Nucleic Acid Delivery Systems on A549 Cell Line

The cytotoxicity of cationic lipid liposomes prepared in Examples 23 and 24 on A549 cells was evaluated in accordance with the same method as Example 28-1.

### Example 28-3

### Toxicity Evaluation of Cationic Lipid-Containing Nucleic Acid Delivery Systems on 293T Cell Line

The cytotoxicity of cationic lipid liposomes prepared in Examples 23 and 24 on 293T cells was evaluated in accordance with the same method as Example 28-1.

FIG. 4 shows that the complexes of siRNA with the cationic lipid-containing liposomes prepared in Examples 23 and 24 exhibited no significant cytotoxicity on Hepa 1-6, A549, and 293T cells as compared with the control.

### Example 29

### Stability Evaluation of Cationic Lipid-Containing Nucleic Acid Delivery Systems

2, 4, 8, 12, 16, 20 μL of the cationic liposomes prepared in Comparative examples 1, 2 and Examples 23, 24 and 25, 5 μl, of siRNA, and 0.1% DEPC-containing distilled water (DW) were added to Eppendorf tubes. These materials were slowly pipetted, mixed and allowed to stand at room temperature for 20 min. The prepared complexes were mixed with loading dye and run on an EtBr-containing 1.5% agarose gel for electrophoresis. The bands were visualized and images were acquired using a UV imager (Gel-doc, Bio-rad, USA). As a result, it can be seen that 12 μL of the cationic lipid-containing liposomes and siRNA formed 100% complexes together, and the stability evaluation of cationic lipid-containing nucleic acid delivery systems was carried out at the above concentration according to the following method.

12 μL of the cationic liposomes prepared in Comparative examples 1, 2 and Examples 23, 24 and 25, 5 μL of siRNA, and 0.1% DEPC-containing distilled water (DW) were added to Eppendorf tubes. These materials were slowly pipetted, mixed and allowed to stand at room temperature for 20 min. The prepared complexes were mixed with fetal bovine serum in a ratio of 1:1 and allowed to stand at 37° C. for 0, 0.5, 1, 3, 6, 12, 24 hr. Then, the mixtures were treated with 0.5% SDS and allowed to stand at 37° C. for 10 min, and mixed with loading dye and run on an EtBr-containing 1.5% agarose gel for electrophoresis. The bands were visualized and images were acquired using a UV imager (Gel-doc, Bio-rad, USA).

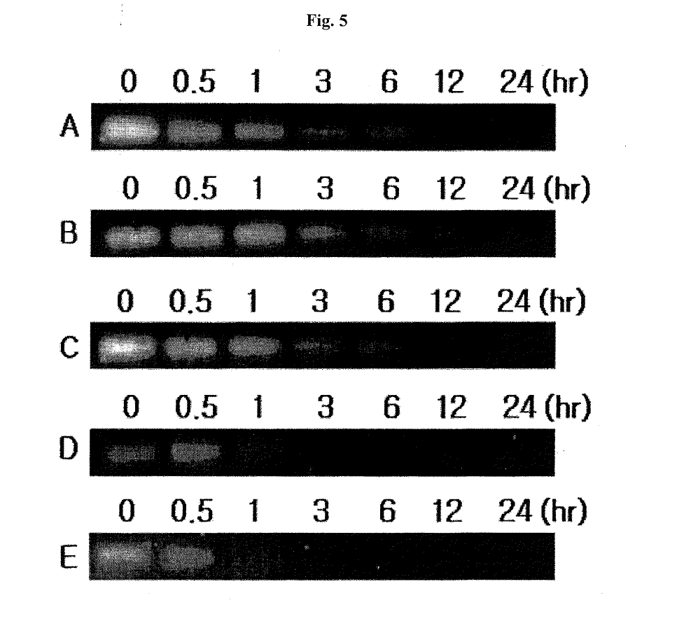

FIG. 5 shows stability experiment results for individual complexes of ribonucleic acid with liposomes (A, B, C) comprising the cationic lipids of the present invention prepared in Examples 23, 24, 25 and complexes of ribonucleic acid with liposomes (D, E) prepared in Comparative examples 1, 2 in serum. From the results of FIG. 5, siRNA was observed even after 12 or 24 hr in the liposome (A) containing the cationic lipid of the present invention and the liposome (B) containing the PEG-conjugated cationic lipid of the present invention, but siRNA was not observed at 3 hr in the liposomes containing the conventional cationic lipid and the PEG-conjugated lipid (PEG-DSPE). Accordingly, it can be seen that the stability of the liposome containing the cationic lipid or PEG-conjugated cationic lipid prepared in the present invention was excellent.

Although the present invention has been illustrated and described with reference to the above embodiments of the present invention, the present invention is not limited to such embodiments. Those skilled in the art can make various modifications and variations to the present invention without departing from the spirit and scope of the present invention and it may be understood that the modifications and variations fall within the scope of the present invention.

1. A cationic lipid represented by the following Formula (I): 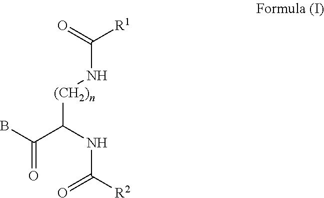
wherein n is 1 to 4, each of R¹ and R² is independently C7-C24 alkyl or alkenyl chain, and B is A-NH, wherein A is a sugar or represented by the following Formula (II), 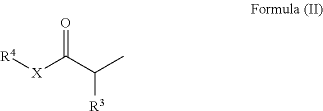
wherein X is NH or O, R³ is a hydrocarbon group having a cationic group derived from an amino acid and represented by the following Formulas (a), (b) and (c), 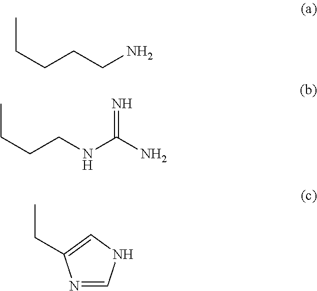
and R⁴ is a ligand and is alkyl, benzyl, a sugar, an antibody, polyethylene glycol, polypropylene glycol, or polyoxyethylene.

2. The cationic lipid according to claim 1, wherein each of R¹ and R² is independently saturated or unsaturated hydrocarbon chain derived from stearate, laurate, myristate, palmitate, or oleate.

3. The cationic lipid according to claim 1, wherein R⁴ is methyl, ethyl, propyl, isopropyl, n-butyl, or benzyl.

4. The cationic lipid of claim 1, wherein the ligand is mPEG (methoxy end-capped polyethylene glycol), polypropylene glycol, or polyoxyethylene.

5. The cationic lipid of claim 1, wherein the ligand is at least one sugar selected from the group consisting of mannitol, sorbitol, xylitol, glucitol, dulcitol, inositol, arabinitol, arabitol, galactitol, iditol, alitol, fructose, sorbose, glucose, mannose, xylose, trehalose, allose, dextrose, altrose, gulose, idose, galactose, talose, ribose, arabinose, lyxose, sucrose, maltose, lactose, lactulose, fucose, rhamnose, melezitose, maltotriose, and raffinose.

6. A delivery system having the ability to transition into cells, comprising a cationic lipid represented by the following Formula (I): 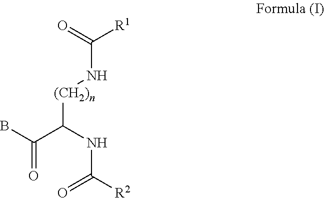
wherein n is 1 to 4, each of R¹ and R² is independently C7-C24 alkyl or alkenyl chain, and B is OH or A-NH, wherein A is a sugar or represented by the following Formula (II), 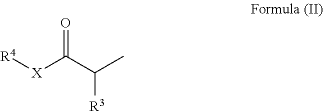
wherein X is NH or O, R³ is a hydrocarbon group having a cationic group derived from an amino acid and represented by the following Formulas (a), (b) and (c), 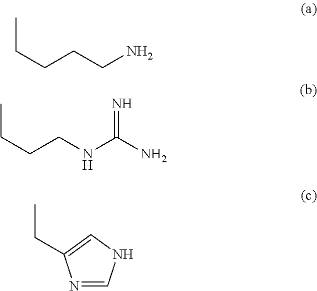
and R⁴ is a ligand and is alkyl or alkenyl, benzyl, a sugar, an antibody, polyethylene glycol, polypropylene glycol, or polyoxyethylene.

7. The delivery system according to claim 6, wherein each of R¹ and R² is independently saturated or unsaturated hydrocarbon chain derived from stearate, laurate, myristate, palmitate, or oleate.

8. The delivery system according to claim 6, wherein R⁴ is methyl, ethyl, propyl, isopropyl, n-butyl, or benzyl.

9. The delivery system of claim 6, wherein the ligand is mPEG (methoxy end-capped polyethylene glycol), polypropylene glycol, or polyoxyethylene.

10. The delivery system of claim 6, wherein the ligand is at least one sugar selected from the group consisting of mannitol, sorbitol, xylitol, glucitol, dulcitol, inositol, arabinitol, arabitol, galactitol, iditol, alitol, fructose, sorbose, glucose, mannose, xylose, trehalose, allose, dextrose, altrose, gulose, idose, galactose, talose, ribose, arabinose, lyxose, sucrose, maltose, lactose, lactulose, fucose, rhamnose, melezitose, maltotriose, and raffinose.

11. The delivery system of claim 6, comprising a drug or nucleic acid as a target material of intracellular or in vivo delivery.

12. The delivery system according to claim 11, wherein the nucleic acid is at least one selected from the group consisting of DNAs, RNAs, aptamers, siRNAs, miRNAs, and antisense oligonucleic acids.

13. The delivery system according to claim 11, wherein the drug is at least one selected from the group consisting of ceftriaxone, ketoconazole, ceftazidime, oxaprozin, albuterol, valacyclovir, urofollitropin, famciclovir, flutamide, enalapril, mefformin, itraconazole, buspirone, gabapentin, fosinopril, tramadol, acarbose, lorazepan, follitropin, glipizide, omeprazole, fluoxetine, lisinopril, tramsdol, levofloxacin, zafirlukast, interferon, growth hormone, interleukin, erythropoietin, granulocyte stimulating factor, nizatidine, bupropion, perindopril, erbumine, adenosine, alendronate, alprostadil, benazepril, betaxolol, bleomycin sulfate, dexfenfluramine, diltiazem, fentanyl, flecainid, gemcitabine, glatiramer acetate, granisetron, lamivudine, mangafodipir trisodium, mesalamine, metoprolol fumarate, metronidazole, miglitol, moexipril, monteleukast, octreotide acetate, olopatadine, paricalcitol, somatropin, sumatriptan succinate, tacrine, verapamil, nabumetone, trovafloxacin, dolasetron, zidovudine, finasteride, tobramycin, isradipine, tolcapone, enoxaparin, fluconazole, lansoprazole, terbinafine, pamidronate, didanosine, diclofenac, cisapride, venlafaxine, troglitazone, fluvastatin, losartan, imiglucerase, donepezil, olanzapine, valsartan, fexofenadine, clacitonin, ipratropium bromide, adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, nicardipine hydrochloride, zolpidem tartrate, amlodipine besylate, ethinyl estradiol, omeprazole, rubitecan, amlodipine besylate/benazepril hydrochloride, etodolac, paroxetine hydrochlroride, atovaquone, podofilox, betamethasone dipropionate, pramipexole dihydrochloride, vitamin, quetiapine fumarate, candesartan, cilexetil, ritonavir, busulfan, carbamazepine, flumazenil, risperidone, carbemazepine, carbidopa, levodopa, ganciclovir, saquinavir, amprenavir, carboplatin, glyburide, sertraline hydrochloride, rofecoxib carvedilol, halobetasol propionate, sildenafil citrate, celecoxib, chlorthalidone, imiquimod, simvastatin, citalopram, ciprofloxacin, irinotecan hydrochloride, sparfloxacin, efavirenz, cisapride monohydrate, tamsulosin hydrochloride, mofafinil, azithromycin, clarithromycin, letrozole, terbinafine hydrochloride, rosiglitazone maleate, diclofenac sodium, lomefloxacin hydrochloride, tirofiban hydrochloride, telmisartan, diazapam, loratadine, toremifene citrate, thalidomide, dinoprostone, mefloquine hydrochloride, trandolapril, docetaxel, mitoxantrone hydrochloride, tretinoin, triamcinolone acetate, estradiol, nelfinavir mesylate, indinavir, beclomethasone dipropionate, famotidine, nifedipine, prednisone, cefuroxime, lorazepam, digoxin, lovastatin, griseofulvin, naproxen, ibuprofen, isotretinoin, tamoxifen citrate, nimodipine, amiodarone, and alprazolam.

14. The delivery system according to claim 11, wherein the drug is an anticancer agent, and the anticancer agent is at least one selected from the group consisting of paclitaxel, vinblastine, adriamycin, oxaliplatin, cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin, phenesterine, vincristine, tamoxifen, dasatinib, piposulfan, maytansinoid, taxanes and CC-1065.

15. A method for preparing a cationic lipid of the following Formula (I), comprising (a) protecting an amine group (—NH₂) of an amino acid having a positive charge with a protecting group; (b) deprotecting the protected amine group to activate the amine group of the amino acid; and (c) binding a carbonyl group of a fatty acid halide to the activated amine group: 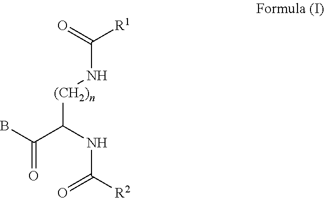
wherein n is 1 to 4, each of R¹ and R² is independently C7-C24 alkyl or alkenyl chain, and B is OH or A-NH, wherein A is a sugar or represented by the following Formula (II), 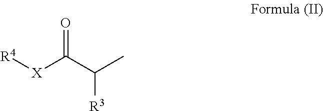
wherein X is NH or O, R³ is a hydrocarbon group having a cationic group derived from an amino acid and represented by the following Formulas (a), (b) and (c), 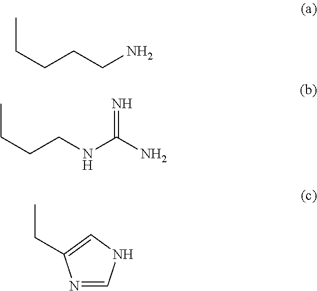
and R⁴ is a ligand and is alkyl, benzyl, a sugar, an antibody, polyethylene glycol, polypropylene glycol, or polyoxyethylene.

16. The method according to claim 15, wherein in the step (a), the amine group (—NH₂) is protected with Boc protecting group using a solution in which tetrahydrofuran is added to t-(Boc)₂O, in the step (b), the protected amine group is deprotected using trifluoroacetate to activate the amine group of the amino acid, and in the step (c), the carbonyl group of the fatty acid halide is combined to the activated amine group using triethylamine.

17. The method according to claim 15, wherein the fatty acid halide is a fatty acid chloride.

18. The method according to claim 15, wherein each of R¹ and R² is independently saturated or unsaturated hydrocarbon chain derived from stearate, laurate, myristate, palmitate, or oleate.

19. The method of claim 15, wherein to the carboxyl group of the amino acid portion of the cationic lipid, an amine group of another amino acid is additionally combined to form an amide bond, or methyl, ethyl, propyl, isopropyl, n-butyl, benzyl, polyethylene glycol, polypropylene glycol, polyoxyethylene, or a sugar is combined as a ligand, or an amine group of still another amino acid in which methyl, ethyl, propyl, isopropyl, n-butyl, benzyl, polyethylene glycol, polypropylene glycol, polyoxyethylene, or a sugar is combined to the carboxyl group of the still another amino acid as a ligand is combined to form an amide bond.

20. The method of claim 15, wherein at least one sugar selected from the group consisting of mannitol, sorbitol, xylitol, glucitol, dulcitol, inositol, arabinitol, arabitol, galactitol, iditol, alitol, fructose, sorbose, glucose, mannose, xylose, trehalose, allose, dextrose, altrose, gulose, idose, galactose, talose, ribose, arabinose, lyxose, sucrose, maltose, lactose, lactulose, fucose, rhamnose, melezitose, maltotriose, and raffinose is combined as the ligand.
